(19) United States
(12) Patent Application Publication
(10) Pub. No.: US 2021/0009959 A1
(43) Pub. Date: Jan. 14, 2021

(54) METHODS FOR ENHANCING OSTEOGENIC DIFFERENTIATION USING VITAMIN D

(71) Applicants: Ghassem Amoabediny, Tehran (IR); Fatemeh Mokhtari-Jafari, Tehran (IR); Marco N. Helder, Amsterdam (NL); Behrouz Zandieh-Doulabi, Amsterdam (NL); Jenneke Klein-Nulend, Amsterdam (NL); Mohammad Mehdi Dehghan, Tehran (IR)

(72) Inventors: Ghassem Amoabediny, Tehran (IR); Fatemeh Mokhtari-Jafari, Tehran (IR); Marco N. Helder, Amsterdam (NL); Behrouz Zandieh-Doulabi, Amsterdam (NL); Jenneke Klein-Nulend, Amsterdam (NL); Mohammad Mehdi Dehghan, Tehran (IR)

(21) Appl. No.: 16/507,174

(22) Filed: Jul. 10, 2019

(51) Int. Cl. C12N 5/0775; A61K 35/28

(52) U.S. Cl. CPC C12N 5/0667; A61K 35/28; C12N 2501/998; C12N 2506/1384; C12N 2501/165; C12N 2501/999

(57) ABSTRACT

A method of enhancing osteogenic differentiation using vitamin D treatment is disclosed. The method utilizes the combined effect of vitamin D treatment and flow-induced shear stress in a modified perfusion bioreactor to treat the bone defect. The method comprising the steps of: isolating adipose tissue from a subject by liposuction; separating adipose-derived stem cells from the adipose tissue; pre-treating the separated adipose-derived stem cells for a predefined time 20 to 40 minutes with vitamin D3; seeding the pre-treated stem cells onto one or more scaffolds; washing of unattached stem cells from the scaffolds after a predefined time of 20 to 30 minutes; culturing the stem cell seeded scaffold by utilizing a modified perfusion bioreactor to form a tissue-engineered construct, where flow induced shear stress is applied, and implanting the tissue-engineered construct into the subject without a need to obtain autologous bone graft.

### BACKGROUND OF THE INVENTION

Over the last decade, the use of growth factors for osteogenic differentiation received a lot of attention, although recently their further application was limited due to high cost and potential adverse side effects. Earlier, it was found that within the short time frame of the one-step surgical procedure, ex vivo exposure to recombinant human bone morphogenetic protein-2 (rhBMP2) as a growth factor facilitated a pronounced increase in proliferation and acceleration of osteogenic differentiation. However, rhBMP2 is rather expensive, and associated with some adverse effects that threats the patient's health.

The Vitamin D metabolites known as 25-hydroxyvitamin D2 and 25-hydroxyvitamin D3 (collectively referred to as “25-hydroxyvitamin D”) are fat-soluble steroid prohormones to Vitamin D hormones that contribute to the maintenance of normal levels of calcium and phosphorus in the bloodstream. The prohormone 25-hydroxyvitamin D2 is produced from Vitamin D2 (ergocalciferol) and 25-hydroxyvitamin D3 is produced from Vitamin D3 (cholecalciferol) primarily by one or more enzymes located in the liver. The two prohormones also can be produced outside of the liver from Vitamin D2 and Vitamin D3 in certain cells, such as enterocytes, which contain enzymes identical or similar to those found in the liver.

The prohormones are further metabolized in the kidneys into potent hormones. The prohormone 25-hydroxyvitamin D2 is metabolized into a hormone known as 1.alpha.25-dihydroxyvitamin D3; likewise, 25-hydroxyvitamin D3 is metabolized into 1.alpha.25-dihydroxyvitamin D3 (calcitriol). Production of these hormones from the prohormones also can occur outside of the kidney in cells which contain the required enzyme(s).

Current bone tissue engineering strategies lack a safe, efficient, and affordable approach that effectively differentiate stem cells into target cell type with minimal adverse effects and could be used within the time frame of a one-step surgical procedure. Therefore, there is a need for a method to overcome the problem of long-term, high-dose differentiation-stimulated factor administration which decrease the potential of proliferation and osteogenic differentiation and increase the risk of adverse effects. Further, there is need for a safe, efficient, and affordable method for bone regeneration. Further, there is a need of a method or eliminating the need for a second surgery to obtain an autologous bone graft, and thus also reducing potential complications from an additional surgery.

### SUMMARY OF THE INVENTION

One aspect of the present disclosure is directed to a method of enhancing osteogenic differentiation, comprising the steps of: (a) pre-treating a plurality of human adipose stem cells for a predefined time of 20-40 mins with vitamin D3; (b) seeding the pre-treated stem cells onto one or more scaffolds; (c) applying flow-induced shear stress to the stem cell seeded scaffolds; and (d) enhancing, synergistically, the osteogenic differentiation and cell proliferation of the plurality of human adipose stem cells due to a combined effect of vitamin D3 and flow-induced shear stress. In one embodiment, the vitamin D3 is calcitriol. In another embodiment, the plurality of human adipose stem cells is pre-treated with calcitriol of 10 nM for 30 mins.

Another aspect of the present disclosure is directed to a method of enhancing osteogenic differentiation, comprising the steps of: (a) isolating adipose tissue from a subject; (b) separating adipose-derived stem cells from the adipose tissue; (c) pre-treating the separated adipose-derived stem cells for a predefined time of 20 to 40 minutes with vitamin D3; (d) seeding the pre-treated stem cells onto one or more scaffolds; and (e) culturing the stem cell seeded scaffolds by utilizing a modified perfusion bioreactor to form a tissue-engineered construct. In one embodiment, the adipose tissue is a human adipose tissue. In one embodiment, the vitamin D3 is calcitriol, and the plurality of human adipose stem cells is pre-treated with calcitriol of 10 nM for 30 mins. In one embodiment, the scaffold is BCP20/80 scaffold composed of 20% HA (hydroxylapatite) and 80% β-TCP (beta-tricalcium phosphate).

In one embodiment, the bioreactor comprises a chamber including an inlet channel coupled to a peristaltic pump; a flow distributor disposed within the chamber comprises at least five distributor channels, the flow distributor is in fluid communication with the inlet channel; a suction tube; and a syringe filter, wherein the flow distributor is configured to apply flow-induced shear stress. In another embodiment, the step of culturing, comprises applying flow-induced shear stress on the stem cells seeded scaffolds. In one embodiment, the step of pre-treating, comprises: treating the calcitriol treated cells with one or more markers selected from the group consisting of RUNX2, ALP, SPARC, ki-67, OPN, OCN, DMP1, VDR, CYP24, CYP27B1, Endotelin1, VEGF165, and VEGF 189.

Another aspect of the present disclosure is directed to a method of enhancing osteogenic differentiation to treat bone defect with one-step surgical procedure, comprising the steps of: (a) isolating adipose tissue from a subject by liposuction; (b) separating adipose-derived stem cells from the adipose tissue; (c) pre-treating the separated adipose-derived stem cells for a predefined time 20 to 40 minutes with vitamin D3; (d) seeding the pre-treated stem cells onto one or more scaffolds; (e) washing of unattached stem cells from the scaffolds after a predefined time of 20 to 30 minutes; (f) culturing the stem cell seeded scaffold by utilizing a modified perfusion bioreactor to form a tissue-engineered construct; and (g) implanting the tissue-engineered construct into the subject by one-step surgical procedure without a need to obtain an autologous bone graft. In one embodiment, the adipose tissue is a human adipose tissue. In another embodiment, the bioreactor comprises a chamber including an inlet channel coupled to a peristaltic pump; a flow distributor disposed within the chamber comprises at least five distributor channels, the flow distributor is in fluid communication with the inlet channel; a suction tube; and a syringe filter, wherein the flow distributor is configured to after flow-induced shear stress on a culture medium passed through the at least five distributor channels. In one embodiment, the culturing step (step f above) is absent in the above-identified method for enhancing osteogenic differentiation.

In one embodiment, the step of culturing, comprises: applying flow-induced shear stress on the stem cells seeded scaffolds. In another embodiment, the plurality of human adipose stem cells is pre-treated with calcitriol of 10 nM for 30 mins. In one embodiment, a combined effect of vitamin D3 and flow-induced shear stress is configured to synergistically enhance the osteogenic differentiation and cell proliferation of the adipose-derived stem cells. In a related embodiment, the step of pre-treating, comprises: treating the calcitriol treated cells with one or more markers selected from the group consisting of RUNX2, ALP, SPARC, ki-67, OPN, OCN, DMP1, VDR, CYP24, CYP27B1, Endotelin1, VEGF165, and VEGF 189.

According to the present invention, the one-step surgical procedure involving short pre-treatment with calcitriol could only be used to treat small bone defects. In case of large bone defects (thickness >10 mm), short pre-treatment with calcitriol does not suffice to reconstruct the defect and culturing the stem cell seeded scaffold within the modified perfusion bioreactor is required to form the tissue-engineered construct. Hence, two-step surgical procedure is required to treat large bone defects.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 exemplarily illustrates a schematic diagram of one step surgical procedure for bone defects with respect to time according to an embodiment of the present invention.

FIG. 2 exemplarily illustrates a modified perfusion bioreactor according to an embodiment of the present invention.

FIG. 3 exemplarily illustrates a flow distributer of the modified perfusion bioreactor according to an embodiment of the present invention.

FIGS. 4A to 4C exemplarily illustrate the effects of hASCs attachment to scaffolds according to an embodiment of the present invention.

FIGS. 5A and 5B exemplarily illustrate the effects of calcitriol treatment on ALP activity of hASCs according to an embodiment of the present invention.

FIG. 6 exemplarily illustrate the effects of calcitriol treatment on ALP activity according to an embodiment of the present invention.

FIG. 7 exemplarily illustrate the effects of calcitriol treatment on cell distribution and ALP activity using NBT/BCIP according to an embodiment of the present invention.

FIGS. 8A to 8I exemplarily illustrate the effects of calcitriol treatment on osteogenic gene expression of hASCs according to an embodiment of the present invention.

FIG. 9 exemplarily illustrates the effects of dynamic and static pretreated/non-treated hASCs on metabolic activity of hASCs according to an embodiment of the present invention.

FIG. 10 exemplarily illustrates the effects of dynamic and static culture of pretreated/non-treated hASCs on ALP activity of hASCs according to an embodiment of the present invention.

FIGS. 11A to 11I exemplarily illustrate the effects of dynamic and static culture of pretreated/non-treated hASCs on osteogenic and angiogenic differentiation of hASCs according to an embodiment of the present invention.

### DETAILED DESCRIPTION

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The present invention generally relates to osteogenic differentiation, and more particularly relates to a method for enhancing osteogenic differentiation using vitamin D treatment. The use of calcitriol (1,25-dihydroxyvitamin D3) as an accelerator of osteoblast differentiation and mineralization is a topic related to the present disclosure.

A method of enhancing osteogenic differentiation using vitamin D treatment is disclosed. The method further discloses that a pre-treatment of human adipose stem cells (hASCs) with low physiological concentration of 1,25-dihydroxyvitamin D3 induces osteogenesis. Further, a synergistic promotion of hASCs osteogenesis and angiogenesis is demonstrated by integrating the effect of fluid shear stress in a modified perfusion bioreactor with short (20-40 min) 1,25-dihydroxyvitamin D3 treatment.

The method comprising the following steps. At one step, a plurality of human adipose stem cells is pre-treated for a predefined time of 20-40 minutes with 1,25-dihydroxyvitamin D3 (vitamin D3). In one embodiment, the vitamin D3 is calcitriol. In one embodiment, the plurality of human adipose stem cells is pre-treated with calcitriol of 10 nM for 30 minutes. At another step, pretreated stem cells are seeded onto one or more scaffolds. At another step, flow-induced shear stress is applied to the stem cell seeded scaffolds. At another step, the osteogenic differentiation and cell proliferation of the plurality of human adipose stem cells are synergistically enhanced due to a combined effect of vitamin D3 and flow-induced shear stress.

In one embodiment, a method of enhancing osteogenic differentiation is disclosed. At one step, adipose tissue is isolated from a subject or patient. At another step, adipose derived stem cells are separated from the adipose tissue. The adipose tissue is washed, digested and centrifuged to obtain a fresh stromal vascular fraction, which contains adipose stem cells. At another step, the separated adipose-derived stem cells are pre-treated for a predefined time of 20 to 40 minutes with vitamin D3, before seeding into the scaffold. In one embodiment, the 1,25-dihydroxyvitamin D3 of about 10 nM concentration is used for pre-treatment of hASCs. In one embodiment, the plurality of human adipose stem cells is pre-treated with calcitriol of 10 nM for 30 mins.

At another step, the pre-treated stem cells are seeded onto one or more scaffolds. In one embodiment, the scaffold could be a BCP20/80 scaffold. The pre-treated hASCs are allowed to attach with the scaffold for a period of about 30 minutes. After the period of time, the hASCs in the scaffolds are washed off to remove the unattached cells. At another step, the stem cell seeded scaffolds are cultured at a modified perfusion bioreactor to form a tissue-engineered construct. In one embodiment, the adipose tissue is a human adipose tissue.

The modified perfusion bioreactor comprises a chamber, a flow distributer, a suction tube and a syringe filter. The chamber includes an inlet channel coupled to a peristaltic pump. The flow distributor disposed within the chamber comprises at least five distributor channels, the flow distributor is in fluid communication with the inlet channel. The flow distributer is configured to apply flow-induced shear stress. In one embodiment, flow-induced shear stress is applied on the stem cells seeded scaffolds. In one embodiment, after pre-treatment with calcitriol, the calcitriol treated cells with one or more markers selected from the group consisting of RUNX2, ALP, SPARC, Ki-67, OPN, OCN, DMP1, VDR, CYP24, CYP27B1, Endotelin1, VEGF165, and VEGF 189.

In another embodiment, a method of enhancing osteogenic differentiation to treat bone defect with one-step surgical procedure for small bone defects and two-step surgical procedure for large bone defects is disclosed. At one step, adipose tissue is isolated from a subject by liposuction. At another step, adipose-derived stem cells are separated from the adipose tissue. At another step, the separated adipose-derived stem cells are pre-treated for a predefined time 20 to 40 minutes with vitamin D3. At another step, the pre-treated stem cells are seeded onto one or more scaffolds. At another step, unattached stem cells are washed from the scaffolds after a predefined time of 20 to 30 minutes. At another step, the stem cell seeded scaffold are cultured at a modified perfusion bioreactor to form a tissue-engineered construct. At another step, the tissue-engineered construct is implanted into the subject without a need to obtain an autologous bone graft.

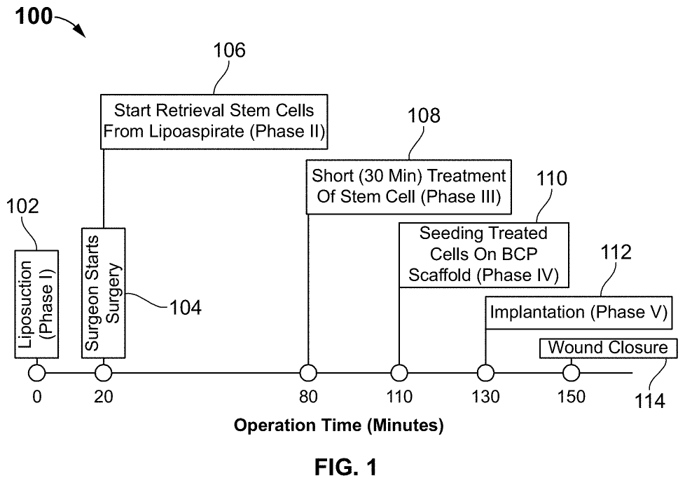

Referring to FIG. 1, a schematic diagram 100 of one step surgical procedure for bone defects to stimulates human adipose stem cells (hASCs) to gain osteogenic phenotype. At block 102, adipose tissue is isolated from the patient by liposuction process. At block 104, a doctor or surgeon starts surgical procedure. At block 106, the adipose stem cells are retrieved from the adipose tissue isolated from the patient. The adipose tissue is washed, digested and centrifuged to obtain a fresh stromal vascular fraction, which contains adipose stem cells. At block 108, the retrieved stem cells are treated with low physiological concentration of 1,25-dihydroxyvitamin D3 (vitamin D3 or calcitriol) for a predefined period of time, before seeding into one or more scaffolds. In one embodiment, the 1,25-dihydroxyvitamin D3 of about 10 nM concentration is used for pre-treatment of hASCs. In one embodiment, the hASCs are pre-treated with low concentrated 1,25-dihydroxyvitamin D3 for a period of about 20-40 minutes. In one embodiment, the hASCs are pre-treated with low concentrated 1,25-dihydroxyvitamin D3 for a period of about 30 minutes (short treatment).

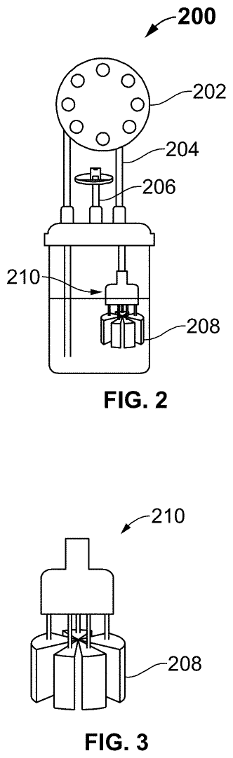

At block 110, the pre-treated hASCs are seeded into the scaffolds. In one embodiment, the scaffold could be a BCP20/80 scaffold. The pre-treated hASCs are allowed to attach with the scaffolds for a period of about 30 minutes. After 30 minutes, the hASCs in the scaffolds are washed off to remove the unattached cells. At block 112, the pre-treated cells are cultured in a modified perfusion bioreactor 200 as shown in FIG. 2, to form a tissue-engineered construct. The bioreactor 200 is configured to apply a flow-induced shear stress to the hASCs. At step 114, the tissue-engineered construct is implanted into the patient by surgical procedure. In one embodiment, the surgical procedure is a one-step surgical procedure. According to the present invention, the one-step surgical procedure involving short pre-treatment with calcitriol could be used to treat small bone defects. In case of large bone defects (thickness >10 mm), short pre-treatment with calcitriol does not suffice to reconstruct the defect and culturing the stem cell seeded scaffold within the modified perfusion bioreactor 200 is required to form the tissue-engineered construct. Hence, two-step surgical procedure is required to treat large bone defects.

Referring to FIG. 2, the system design of modified perfusion bioreactor 200 is disclosed, according to an embodiment of the present invention. The modified perfusion bioreactor 200 comprises a peristaltic pump 202, a silicon tube 204, a syringe filter of 0.2 micron 206, one or more scaffolds 208 and a flow distributer 210. The chamber of modified perfusion bioreactor 200 comprises an inlet channel. The inlet channel is in fluid communication with the flow distributor 210. The flow distributer 210 comprises at least five distributor branches or channels 208. The peristaltic pump 202 enables the flow of culture medium via tubing 204. In one embodiment, the peristaltic pump 202 operates with the flow rate of about 5 ml/min, where the pump 202 has the flow rate of about 1 ml/min for each scaffold.

Referring to FIG. 3, the flow distributer 210 of the modified perfusion bioreactor 200 is disclosed, according to an embodiment of the present invention. The flow distributer 210 distributes the culture medium used in the modified perfusion bioreactor 200 among scaffolds. In one embodiment, the flow distributer 210 is divided into five branches 208 to distribute the culture medium into five scaffolds. In one embodiment, the scaffolds are PLGA/β-TCP scaffolds. The five-branch flow distributer 210 distributes the flow of culture medium to the scaffolds via inlet channels. The distribution in the flow culture medium creates a flow-induced shear stress. The combined effect of flow-induced shear stress and 1,25-dihydroxyvitamin D3 promotes the osteogenic and angiogenic synergy of hASCs. The combined effect promotes the osteogenic and angiogenic synergy of hASCs for regeneration of bone defects, especially for large bone defects.

In one embodiment, a method of static culture of hASCs by pre-treatment with vitamin D3 (calcitriol) and attachment of hASCs to the scaffold used in one-step surgical procedure comprises the following steps. At one step, isolating hASCs as explained in FIG. 1. In an embodiment, the hASCs from three donors at passage 3 are used for further processing. As an optional step, the obtained hASCs are either or not incubated for a period of about 30 mins with low concentrated calcitriol of about 10 nM at room temperature.

At next step, the calcitriol treated hASCs are washed twice with Phosphate Buffered Saline (PBS) to remove calcitriol. Further, the washed hASCs are centrifuged and resuspended in Dulbecco's Minimum Essential Medium (DMEM) without any supplements. At another step, the resuspended cells are seeded on the scaffold and allowed to attach to the scaffolds for a period of about 30 minutes. At next step, the scaffolds with attached cells are transferred to 12-well plates with Costar® Transwell® containers (Corning Life Sciences, Lowell, Mass., USA) containing expansion medium (DMEM) supplemented with 10% Fetal Clone I (FCI), antibiotics (1% penicillin/streptomycin/fungizone (PSF)), 50 μM ascorbic acid (Merck, Darmstadt, Germany), and 10 mM β-glycerol phosphate (Merck, Darmstadt, Germany) (PLGA/β-TCP). At next step, the hASCs seeded scaffolds are incubated at 5% CO₂ in a humidified incubator at predefined temperature of about 37° C. for a period of about 3 weeks.

In one embodiment, dynamic culture of pre-treated hASCs with calcitriol, using the modified perfusion bioreactor comprises the following steps. At one step, the scaffolds seeded with hASCs are transferred to the modified perfusion bioreactor 200 as shown in FIG. 2. In one embodiment, the scaffolds are 3D PLGA/β-TCP. The modified perfusion bioreactor 210 comprises five scaffolds, loaded with a total of 70 ml of culture medium as shown in FIG. 2. At another step, 30 ml of the culture medium is replaced twice a week for a total period of two weeks. At another step, the culture medium in the modified perfusion bioreactor 200 is distributed to the five scaffolds with the flow rate of about 5 ml/min using the peristaltic pump 202.

In one embodiment, the method of static culture of pre-treated hASCs is continued in a flask bioreactor with the same volume of culture medium utilized in the dynamic culture. Further, the methods of culturing the hASCs are divided into different groups. In one embodiment, the methods of culturing of hASCs are divided into four groups such as, static culture of non-treated hASCs, static culture of pre-treated hASCs with calcitriol, dynamic culture of non-treated hASCs and dynamic culture of pre-treated hASCs with calcitriol.

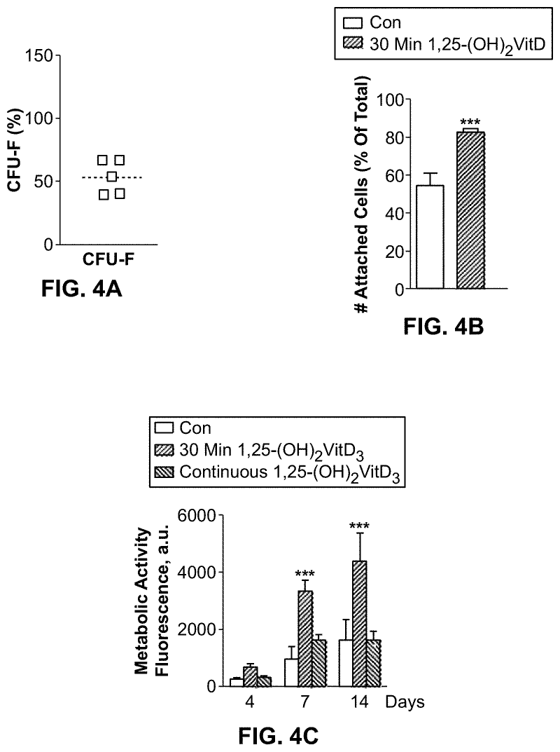

The results of the static culture and dynamic culture of non-treated and pre-treated hASCs are analyzed based on various factors. Referring to FIGS. 4A to 4C, the effect of hASC attachment to the BCP20/80 scaffold with or without calcitriol at day zero and calcitriol treatment effects on metabolic activity of hASCs is disclosed. The number of Colony Forming Unit (CFU) is counted for 14 days after seeding and culturing of non-treated hASCs on tissue culture plastic. FIG. 4A shows the average number of CFU of non-treated hASCs cultured on tissue culture plastic for two weeks is around 53%, which reflects the number of viable hASCs in adipose tissue.

FIG. 4B shows the cell attachment to BCP 20/80 scaffolds after 30 minutes of pre-treatment with 10 nM calcitriol, where the cell attachment is significantly increased when compared to cell attachment in non-treated cells (controls). In one embodiment, the cell attachment of pre-treated hASCs to BCP scaffolds in increased by 1.5 folds, that is, from 54% to 83% when compared to non-treated cells.

The effect of 30 minutes pre-treatment with calcitriol on hASC proliferation is exemplarily illustrated in FIG. 4C. The 30 minutes pre-treatment with calcitriol significantly increases the cell number after 2 and 3 weeks compared to continuous treatment with calcitriol. The 30 minutes pre-treatment with calcitriol increases the cell number at day 14 by 3.5-fold, and at day 21 by 2.6-fold. The continuous treatment of hASCs with calcitriol for 3 weeks increases the cell number only at day 14 by 1.7-fold, but not at day 21 compared to non-treated controls as shown in FIG. 4C. The results of hASC proliferation are expressed as CFU (in %) versus time (in days) and represent means±standard errors of the means of the colon forming unit counts (n=3), where p<0.0001.

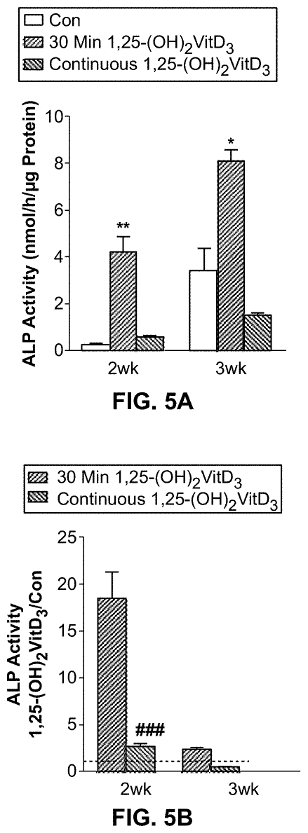

Referring to FIGS. 5A and 5B, the effects of calcitriol treatment on ALP activity of hASCs, where the calcitriol pre-treatment is conducted for a short period of 30 minutes and a long period of about 3 weeks. The 30 minutes calcitriol pre-treatment significantly increases the ALP activity in hASCs after 2 and 3 weeks of culture when compared to continuous calcitriol treatment and non-treated hASCs as shown in FIG. 5A. The 30 minutes incubation with calcitriol increased ALP activity after 2 weeks by 18.5-fold and after 3 weeks by 2.4-fold of culture as shown in FIG. 5B. The ALP activity of hASCs after 30 min pre-treatment with calcitriol is increased by 18.5-fold compared to non-treated cells after 2 weeks of culture. The ALP activity of continuous treated cells with calcitriol is increased by 2.6-fold relative to non-treated cells after 2 weeks of culture.

FIG. 5B shows the continuous treatment with 10 nM calcitriol increases the ALP activity after 2 weeks by 2.6-fold, but not at 3 weeks (i.e., only 0.4-fold). The results of pre-treatment with calcitriol on ALP activity in hASCs seeded in BCP scaffold are expressed as ALP activity (in nmol/h/μg protein) versus time (in weeks) and represent means±standard errors of the means of counts (n=3), where *significantly different from control p<0.05, **p<0.01, ###significantly different from 30 minutes calcitriol, p<0.001.

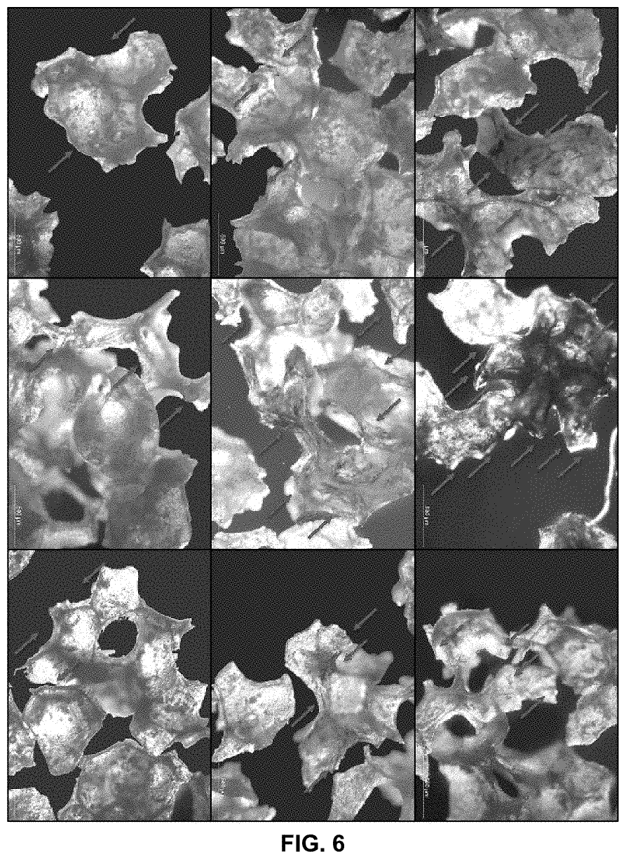

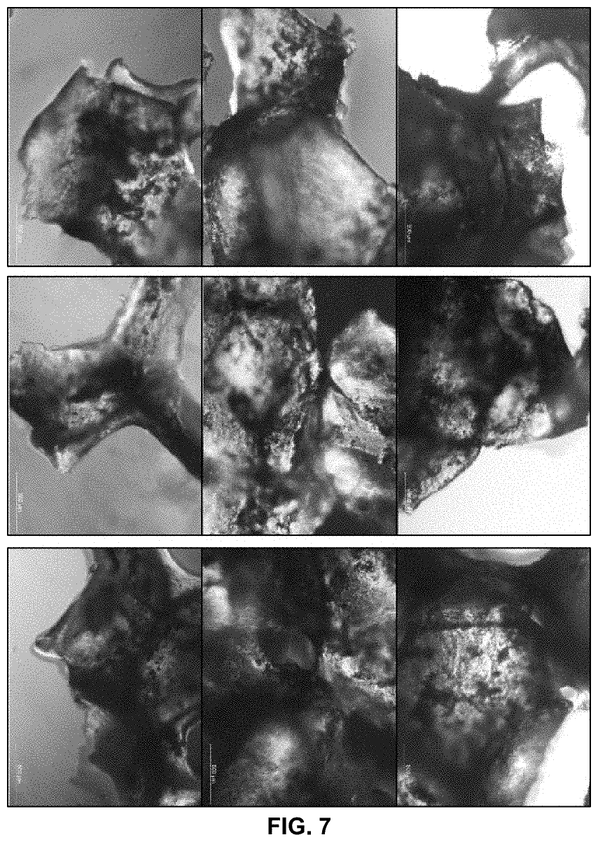

Referring to FIG. 6, the effect of calcitriol treatment on ALP activity in hASCs by ALP staining using nitro blue tetrazolium chloride/5-bromo-4-chloro-3-indolyl phosphate (NBT/BCIP) is disclosed. The 30 minutes calcitriol treatment notably increases the ALP activity after a period of about 2 and 3 weeks of culture compared to the continuous treatment. The increase in ALP activity is confirmed by ALP staining after 14 days of culture. FIG. 7 shows the effects of short calcitriol treatment and long calcitriol treatment on cell distribution and ALP activity using NBT/BCIP. The cell distribution is confirmed by 4′,6-diamidino-2-phenylindole (DAPI) staining.

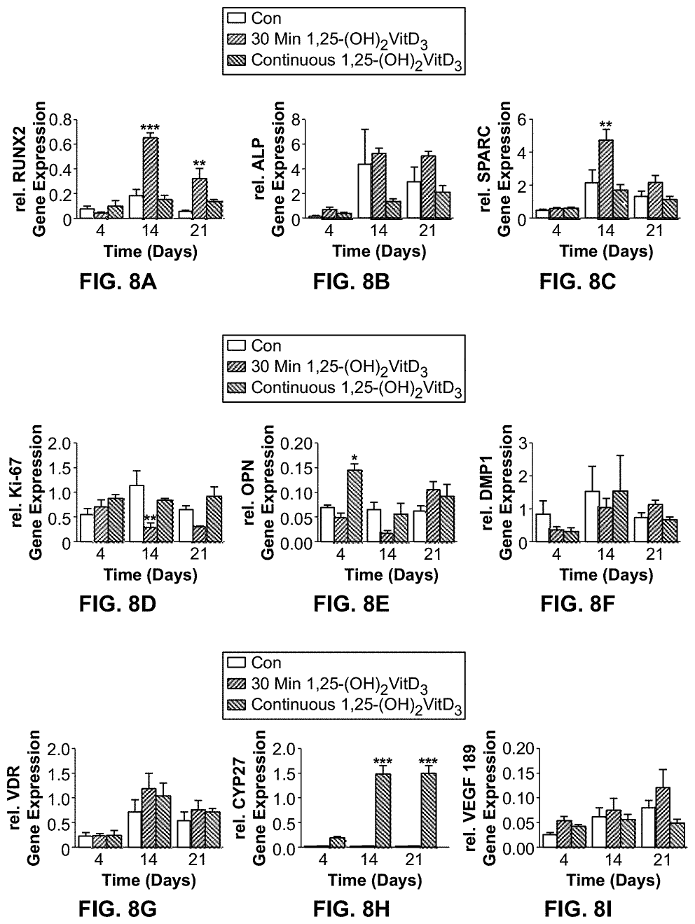

Referring to FIGS. 8A to 8I, the effects of short and long calcitriol treatment on osteogenic gene expression of hASCs is disclosed, where hASCs are seeded in BCP scaffold. The stimulatory effect of 30 min calcitriol on osteogenic gene expression in hASCs seeded on BCP20/80 at day 21 is more noticeable than continuous calcitriol treatment. The 30 mins incubation with calcitriol increases the RUNX2 (early osteogenic marker) gene expression in hASCs by 3.6-fold after 2 weeks, and 5.7-fold after 3 weeks of culture compared to non-treated hASCs. FIGS. 8A to 8I exemplarily illustrate the effects of calcitriol treatment on osteogenic gene expression of hASCs.

The continuous treatment with calcitriol decreases RUNX2 (early osteogenic marker) expression by 0.81-fold after 2 weeks and increased by 2.4-fold after 3 weeks as shown in FIG. 8A. The 30 minutes calcitriol upregulates the ALP (intermediate osteogenic marker) gene expression in hASCs seeded on BCP20/80 as shown in FIG. 8B. The 30 minutes pre-treatment with calcitriol increases the SPARC (late osteogenic marker) expression by 2.1-fold at day 14, while continuous treatment decreases the SPARC expression by 0.8-fold as shown in FIG. 8C.

The 30 minutes pre-treatment of hASCs with calcitriol decreases Ki-67 (proliferation marker) gene expression, but there is no change in continuous calcitriol treatment during 3 weeks of culture as shown in FIG. 8D. The continuous treatment upregulates OPN (intermediate osteogenic marker) gene expression at day 4, whereas 30 minutes calcitriol treatment increases OPN expression at day 21 as shown in FIG. 8E. Also, a gradual and insignificant increase in DMP1 (late osteogenic marker) gene expression is observed over the period of about 3 weeks as shown in FIG. 8F.

FIG. 8G shows the 30 minutes pre-treatment with calcitriol increases vitamin D nuclear receptor (VDR) gene expression in hASCs compared to continuous treatment, with maximal stimulation at day 14. The continuous treatment with calcitriol remarkably increases CYP24 (cytochrome p450-enzyme) gene expression, which is associated with inactivation of vitamin D3 molecule. Further, the CYP24 is not expressed in non-treated controls and in 30 minutes calcitriol pre-treated cells, but significantly increases in continuously treated cells during 3 weeks of culture as shown in FIG. 8H.

The 30 minutes pre-treatment of hASCs with calcitriol increases VEGF189 gene expression, whereas in continuous calcitriol treatment, the VEGF189 gene expression reaches almost steady expression levels during the 3 weeks of culture. The VEGF189 gene expression in 30 minutes calcitriol pre-treated hASCs is increased by 1.5-fold at day 21, but it is decreased in continuous treatment by 0.6-fold compared to non-treated hASCs as shown in FIG. 8I. The results of pre-treatment with calcitriol on osteogenic gene expression of hASCs seeded in BCP scaffold are expressed as gene expression (of various markers) versus time period (in days) and represent means±standard errors of the means of counts (n=3), where *significantly different from control, p<0.05, **p<0.01, ***p<0.001.

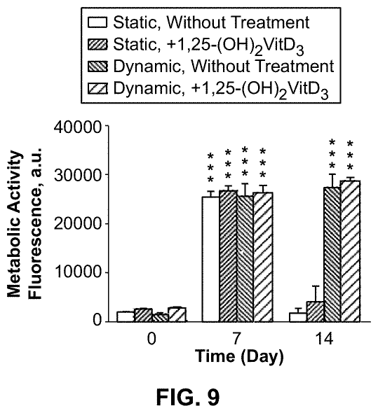

Referring to FIG. 9, the effects of dynamic pre-treated/non-treated hASCs versus static pre-treated/non-treated hASCs on metabolic activity or cell proliferation of hASCs is disclosed, where the pre-treated hASCs are seeded in 3D PLGA/β-TCP scaffold. In one embodiment, the hASCs are pre-treated with or without 10 nM calcitriol for 30 minutes and cultured in the modified perfusion bioreactor 200 (dynamic culture) and flask bioreactor (static culture). The perfusion culture of pre-treated hASCs with calcitriol slightly decelerates the cell proliferation when compared to perfusion culture of non-treated hASCs after 7 and 14 days of culture. After 7 days, the static culture of non-treated hASCs, static culture of pre-treated hASCs, dynamic culture of non-treated hASCs, and dynamic culture of pre-treated hASCs increase cell proliferation by 14.1-fold, 10.6-fold, 15.9-fold, and 9.9-fold relative to the first day (after overnight static culture), respectively. Also, the culturing groups are compared and there is no significant difference between the groups. However, the 14 days of dynamic culture of both non-treated hASCs and pre-treated hASCs increases cell proliferation by 1.1-fold relative to 7 days of culture.

Further, the 14 days of static culture of non-treated hASCs decreases proliferation by 0.9-fold, whereas the static culture of pre-treated hASCs, dynamic culture of non-treated hASCs, and dynamic culture of pre-treated hASCs increase cell proliferation by 1.6-fold, 17-fold, and 10.8-fold relative to the first day, respectively. The result on cell proliferation of hASCs seeded in 3D PLGA/β-TCP scaffold is expressed as metabolic activity versus time period (in days) and represent means±standard errors of the means of counts (n=3), where ***significantly different the first day, p<0.001.

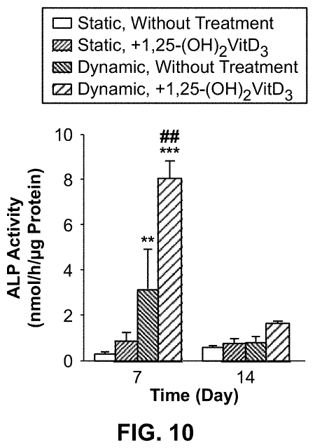

Referring to FIG. 10, the effects of dynamic pre-treated/non-treated hASCs versus static pre-treated/non-treated hASCs on ALP activity of hASCs are disclosed, where the hASCs are seeded in 3D PLGA/β-TCP scaffold. In one embodiment, the hASCs are pre-treated with or without 10 nM calcitriol for 30 minutes and cultured in the modified perfusion bioreactor 200 (dynamic culture) and flask bioreactor (static culture). The ALP activity of hASCs after 7 days perfusion culture of pre-treated hASCs is increased by 9.2-fold compared to static culture of pre-treated hASCs, and the ALP activity of non-treated hASCs is increased by 10.3-fold compared to static culture of non-treated hASCs. The 7 days perfusion culture of pre-treated hASCs increases ALP activity notably by 2.6-fold compared to perfusion culture of non-treated hASCs.

The 14 days of perfusion culture of non-treated hASCs increases ALP activity by 1.4-fold relative to static counterpart, and perfusion culture of pre-treated hASCs increases ALP activity by 2.2-fold relative to static culture of pre-treated hASCs. Moreover, calcitriol pre-treatment increases ALP activity of hASCs by 2.8-fold at day 7 and 1.3-fold at day 14 compared to non-treated hASCs in static condition. On the whole, the highest ALP activity is observed at day 7, which belongs to perfusion culture of pre-treated hASCs with calcitriol. Further, the result of dynamic pre-treated/non-treated hASCs versus static pre-treated/non-treated hASCs on ALP activity of hASCs is expressed as ALP activity (in nmol/h/μg protein) versus time period (in days) and represent means±standard errors of the means of counts (n=3), where **significantly different the static culture counterparts, p<0.01, ***p<0.001, ##significantly different from the dynamic culture of non-treated hASCs.

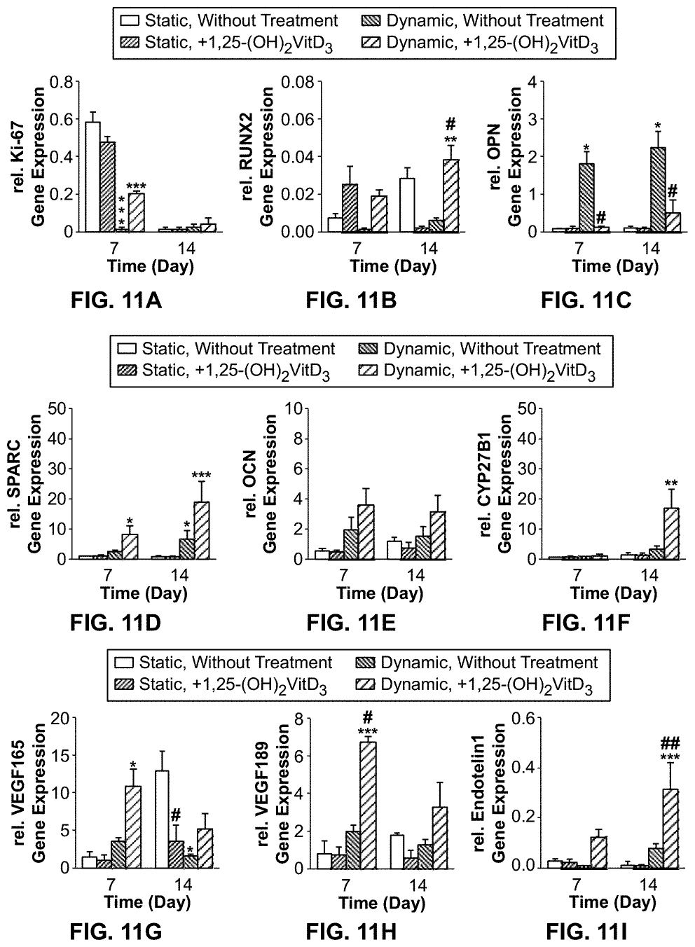

Referring to FIGS. 11A to 11I, the effects of perfusion culture of hASCs on osteogenic and angiogenic differentiation of hASCs are disclosed, where the hASCs are seeded in 3D PLGA/β-TCP scaffold. In one embodiment, the hASCs are pre-treated with or without 10 nM of calcitriol for 30 minutes and cultured in the modified perfusion bioreactor (dynamic culture) and flask bioreactor (static culture). FIGS. 11A to 11I shows the effects of dynamic pre-treated/non-treated hASCs versus static pre-treated/non-treated hASCs on osteogenic and angiogenic differentiation of hASCs. The perfusion culture of pre-treated hASCs increase Ki-67 gene expression by 5.1-fold, RUNX2 gene expression by 82.8-fold, OPN gene expression by 60-fold, SPARC gene expression by 33.2-fold, OCN gene expression by 4.1-fold, CYP27B1 gene expression by 15.4-fold, VEGF165 gene expression by 1.4-fold, VEGF189 gene expression by 5.7-fold and Endotelin-1 gene expression by 35-fold relative to static culture of pre-treated hASCs after 14 days of culture, as shown in FIGS. 11A to 11I respectively.

FIGS. 11A to 11I further illustrates the effect of perfusion culture of pre-treated hASCs, which enhance gene expression of Ki-67 by 1.8-fold, RUNX2 by 6.4-fold, SPARC by 2.8-fold, OCN by 2.1-fold, CYP27B1 by 5.3-fold, VEGF165 by 3.1-fold, VEGF189 by 2.5-fold and Endotelin-1 by 4-fold relative to perfusion culture of non-treated hASCs after 14 days of culture, respectively. Also, on day 7, the dynamic culture of pre-treated hASC increases the gene expression of OPN by 47.3-fold (0.108438/0.002291), SPARC by 7.5-fold, OCN by 7.1-fold, VEGF165 by 9.8-fold, VEGF189 by 8.8-fold and Endotelin-1 by 5.4-fold relative to static culture of treated hASC, and enhance the gene expression of Ki-67 by 45.9-fold, RUNX2 by 186.5-fold, SPARC by 3-fold, OCN by 1.8-fold, VEGF165 by 3-fold, VEGF189 by 3.4-fold, and Endotelin-1 by 120.9-fold relative to perfusion culture of non-treated hASCs.

The results of dynamic pre-treated/non-treated hASCs versus static pre-treated/non-treated hASCs on osteogenic and angiogenic differentiation of hASCs is expressed as gene expression versus time period (in days) and represent means±standard errors of the means of counts (n=3), *significantly different from control, p<0.05, **p<0.01, ***p<0.001, #significantly different from control, p<0.05, ##p<0.01.

Based on the above listed analysis, the hASCs shows differential responses after pre-treatment of hASCs for 30 minutes with 10 nM calcitriol. More specifically, it is observed that (a) the calcitriol pre-treated hASCs adheres significantly to BCP20/80 scaffolds compared to non-treated control hASCs, (b) the proliferation and several osteogenic differentiation markers (ALP activity, RUNX2 and SPARC gene expression) are significantly enhanced by 30 minutes calcitriol pre-treatment when compared to control treatment, (c) the 30 minutes calcitriol pre-treatment effects on osteogenic differentiation appears far more noticeable compared to continuous treatment with calcitriol, and (d) the 30 minutes pre-treatment with calcitriol may contribute to the promotion of angiogenesis.

From the above results, it is found that the rapid attachment of hASCs to the BCP scaffolds is in line with previous findings of different culture groups for other classes of scaffolds consisting of polymeric, collagenous (20), β-TCP, and BCP20/80 biomaterials (slightly higher attachment rate compared to β-TCP). The obtained data indicates a significantly higher attachment rate of the pre-treated hASCs on BCP scaffolds (1.5-fold) versus non-treated hASCs. Hence, the 30 minutes pre-treatment with calcitriol appears superior to BMP2, which may benefit the one-step surgical procedure.

Further, the increase in cell proliferation of 30 minutes calcitriol pre-treated hASCs after 2 and 3 weeks of incubation is noticeable. On the other hand, 3 weeks of continuous treatment significantly decreases the proliferation rate, which agrees with findings by using primary rat osteoblasts. Therefore, the enhancement of cell proliferation through 30 minutes pre-treatment with calcitriol seems promising for implantation in vivo due to enhanced extracellular matrix formation and consequently bone formation. Also, the effect of 30 minutes calcitriol pre-treatment on osteogenic differentiation and ALP activity is more noticeable after 14 days of culture when compared to 4 and 21 days of culture, which indicates a time-dependency of stimulation of hASCs by calcitriol (1,25-(OH)2VitD3).

Further, the results of continuous treatment with calcitriol are also reported in other studies on MC3T3-E1, primary rat osteoblasts, and mesenchymal stem cells derived from human alveolar periosteum, which are in agreement with the current data. However, the current result shows that the following 14 days of incubation sufficiently increase the ALP activity in hASCs pre-treated with calcitriol for 30 minutes when compared to continuously treatment of hASCs with calcitriol.

Most of the biological actions of 1,25-(OH)2VitD3, including cell proliferation and differentiation, are considered to be exerted through the VDR-mediated control of target genes. Moreover, silencing VDR causes a significant decrease in mineralized bone volume after calcitriol treatment. The VDR gene expression is slightly higher in 30 minutes pre-treated hASCs, but no significant differences among groups are found. However, the strong upregulation of CYP24 gene expression in the continuously stimulated, but not in the 30 min-stimulated and control groups presents an alternative explanation. Thus, the upregulation of the CYP24 gene product may result in the inactivation of calcitriol as a consequence of long-term treatment with 1,25-(OH)2VitD3. The 30 minutes pre-treatment with calcitriol enhances the VEGF189 expression. The VEGF189 stimulates endothelial cell proliferation and migration in vitro, and contributes to the promotion of angiogenesis. The expression of VEGF correlates with osteoblastic differentiation, and is low when osteoblastogenesis begins, and it peaks during mineralization. The continuous treatment (21 days) adversely affects the VEGF189 gene expression in hASCs to a level even below that in non-treated hASCs.

In addition, the present invention examines the combined effect of flow-induced shear stress by means of the modified perfusion bioreactor and calcitriol pre-treatment, which could benefit the osteogenic and angiogenic surgery of human adipose stem-cells (hASCs). Based on the above examination, it is found that (a) the pre-treated hASCs with calcitriol has slightly lower proliferation compared to non-treated hASC in both static and dynamic culture, (b) the perfusion culture of pre-treated hASCs with calcitriol significantly increases the ALP activity at day 7 compared to other cultures, (c) the perfusion culture for 7 days enhances the angiogenic differentiation, although osteogenic differentiation of hASCs is further promoted by 14 days of bioreactor culture, and (d) the synergistic promotion of osteogenic and angiogenic differentiation of hASCs is achieved by combining the effect of flow-induced shear stress by means of a modified perfusion bioreactor and calcitriol pre-treatment.

Although there is no significant difference in cell proliferation between the groups after 7 days of culture, cell proliferation of static cultures significantly decreases after 14 days. The proliferation of static cultures could be identified by (a) high cell proliferation on the surface of the scaffold, (b) subsequent blockage in the pores of outer surface, (c) consequent inhibition of oxygen, (d) nutrient delivery to the central region of the scaffold and (e) accumulation of metabolites which also limits cell proliferation inside the scaffold. The results of enhanced ALP activity under dynamic cultivation are reported in other studies on human embryonic stem cell-derived mesenchymal progenitors using modular perfusion bioreactor which are in agreement with the current data. From this analysis, the osteogenic differentiation is enhanced in bioreactor culture, especially after 14 day of culture, with upregulation of RUNX2, SPARC, and OCN gene expressions compared to static conditions, which is also demonstrated by Burgio et al., in which ALP, collagen I and OCN gene expression of human bone marrow mesenchymal stromal cells (hBMSCs) are upregulated during cultivation in a perfusion flow bioreactor.

The RUNX2 gene expression is a well-known master transcriptional regulator of skeletogenesis and strongly expressed by preosteoblasts and immature osteoblasts. The RUNX2 gene expression is significantly promoted by the combined effect of perfusion culture and calcitriol pre-treatment, while it is not pronounced in perfusion culture alone or calcitriol pre-treatment alone. The results validate the synergistic effect of flow-induced shear stress and calcitriol pre-treatment. The OPN gene expression is crucial for bone remodeling and biomineralization, which is upregulated in dynamic cultures of non/pre-treated hASCs, especially in non-treated hASCs.

The SPARC gene expression (as an early skeletogenic factor) is also upregulated by perfusion culture of pre-treated hASCs. The enhanced OCN gene expression is regarded (as an established late marker) for osteoblast differentiation and appears concomitantly with the mineralization phase of bone formation, in perfusion culture of pre-treated hASCs may be indicative of an increased number of mature osteoblasts within the construct. The observations regarding combined effect of fluid-induced shear stress and calcitriol pre-treatment may be ascribed to the upregulation of CYP27B1, which regulates cellular proliferation and differentiation, and elevates the levels of calcitriol associated with increased expression of CYP27B1.

The expression of VEGF correlates with osteoblastic differentiation (i.e., osteoblastogenesis upregulated when VEGF gene expression downregulated), which is consistent with the data collected from the results. It shows that 7 days of hASC dynamic culture by means of modified perfusion bioreactor enhances VEGF189, VEGF165, and Endothelin-1 gene expression, which is in line with recent findings of Burgio et al. using hBMSCs cultivated in a perfusion flow bioreactor, and Nguyen et al. using human mesenchymal stem cells (hMSC) and endothelial cells cultured in a tubular perfusion bioreactor. It stimulates that scaffolds cultured in the bioreactor for 7 days exhibits a trend for greater angiogenic gene expression than 14 days, which is also agreed with reported data by Mitra et al. in terms of greater vessel density. Also, it is found that VEGF189, VEGF165, and Endothelin-1 genes in perfusion culture of pre-treated hASCs are expressed at a higher value than in perfusion culture of non-treated hASCs and static cultures, which signifies the heightened angiogenic potential of perfusion culture of pre-treated hASCs.

Therefore, the present invention reveals that 30 minutes incubation with the low physiological dose of calcitriol (10 nM) is sufficient to promote cell attachment to BCP20/80 scaffolds compared to non-treated cells. It is also sufficient to enhance proliferation as well as stimulate hASCs to gain an osteogenic phenotype in vitro when cultured on BCP20/80 scaffolds. Furthermore, the short pre-treatment (for 30 minutes) with calcitriol is expected to promote angiogenesis in bone tissue-engineered constructs. The results from the above analysis indicate that a short calcitriol pre-treatment is a promising tool for use in a clinical one-step surgical procedure. These results could be extrapolated and implemented in future developments of treatment strategies for large bone defects.

In addition, alamarBlue assay, ALP assay, ALP staining, analysis of osteogenic and angiogenic gene expression and related protein content to evaluate the synergistic effects of flow-induced shear stress using a modified perfusion bioreactor and calcitriol pre-treatment are used for the examinations.

As compared to bioreactor culture of non-treated hASCs, static culture of non-treated hASCs and static culture of pre-treated hASCs, the bioreactor culture of pre-treated hASCs enhance the ALP activity, osteogenic gene markers include RUNX2, SPARC, and OCN, especially on day 14, as well as angiogenic gene markers include VEGF165, VEGF189, notably on day 7 and Endotelin-1 in particular on day 14. The significant upregulation of VEGF165, VEGF189, Endotelin-1 hints strongly at a synergy between perfusion culture and calcitriol pre-treatment which improved angiogenesis. The synergistic promotion of osteogenic and angiogenic differentiation of hASCs by combining the effect of fluid shear stress in a modified perfusion bioreactor with short (30 min) 1,25-dihydroxyvitamin D3 treatment offers a promising biomimetic solution for bone tissue engineering that improves bone reconstruction.

The protocol of short calcitriol treatment is cost effective and no risk to the health of the patient. The hASCs are stimulated to gain the osteogenic phenotype and promotes proliferation. The method of the present invention improves the efficiency and effectiveness of the tissue-engineered bone in bone repair. Also, the method overcomes the inherent adverse effects with growth factors. Further, the method of short calcitriol treatment could be applied immediately after the isolation of adipose tissue from the patient and cell extraction, which allows one-step surgical procedure for small bone defects, thereby avoiding expensive cell culture procedures and another surgical operation. The ex vivo exposure for 20-40 minutes to the low physiological concentration (10 nM) of 1,25-dihydroxyvitamin D3 (calcitriol) increases the cell proliferation and osteogenic differentiation significantly greater than continuous treatment with calcitriol.

The present invention provides a safe, efficient and affordable method for bone regeneration. The present invention further demonstrates that the short pre-treatment (20-40 minutes) of hASCs with physiological concentration (10 nM) of calcitriol, fitting within the procedural time frame, suffices to induce osteogenesis, and is far superior to continuous treatment in stimulating proliferation and osteogenic differentiation of hASCs. Moreover, the combined effect of short calcitriol treatment and flow-induced shear stress using a modified perfusion bioreactor promote osteogenesis and angiogenesis synergy of human adipose stem cells (hASCs), which could contribute to the survival of engineered constructs in large bone defects (>1 cm). Also, the method eliminates the need for a second surgery to obtain an autologous bone graft, and thus also reduces the potential complications from the additional surgery.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions.

Although specific terms may be employed herein, they are used only in generic and descriptive sense and not for purposes of limitation. Accordingly, the present invention is not limited to the specific embodiments illustrated herein. While the above is a complete description of the preferred embodiments of the invention, various alternatives, modifications, and equivalents may be used. Therefore, the above description and the examples should not be taken as limiting the scope of the invention, which is defined by the appended claims.

1. A method of enhancing osteogenic differentiation, comprising the steps of:
pre-treating a plurality of human adipose stem cells for a predefined time of 20-40 mins with vitamin D3;
seeding the pre-treated stem cells onto one or more scaffolds;
applying flow-induced shear stress to the stem cell seeded scaffolds, and
enhancing, synergistically, the osteogenic and angiogenic differentiation of the plurality of human adipose stem cells due to a combined effect of vitamin D3 and flow-induced shear stress.

2. The method of claim 1, wherein the vitamin D3 is calcitriol.

3. The method of claim 1, wherein the plurality of human adipose stem cells is pre-treated with calcitriol of 10 nM for 30 mins.

4. A method of enhancing osteogenic differentiation, comprising the steps of:
isolating adipose tissue from a subject;
separating adipose-derived stem cells from the adipose tissue;
pre-treating the separated adipose-derived stem cells for a predefined time of 20 to 40 minutes with vitamin D3;
seeding the pre-treated stem cells onto one or more scaffolds, and
culturing the stem cell seeded scaffolds by utilizing a modified perfusion bioreactor to form a tissue-engineered construct.

5. The method of claim 4, wherein the adipose tissue is a human adipose tissue.

6. The method of claim 4, wherein the vitamin D3 is calcitriol.

7. The method of claim 4, wherein the plurality of human adipose stem cells is pre-treated with calcitriol of 10 nM for 30 mins.

8. The method of claim 4, wherein the scaffolds is BCP20/80 scaffold.

9. The method of claim 4, wherein the bioreactor comprises:
a chamber including an inlet channel coupled to a peristaltic pump;
a flow distributor disposed within the chamber comprises at least five distributor channels, the flow distributor is in fluid communication with the inlet channel;
a suction tube, and
a syringe filter, wherein the flow distributor is configured to apply flow-induced shear stress.

10. The method of claim 4, wherein the step of culturing, comprises: applying flow-induced shear stress on the stem cells seeded scaffolds.

11. The method of claim 4, wherein the step of pre-treating, comprises: treating the calcitriol treated cells with one or more markers selected from the group consisting of RUNX2, ALP, SPARC, ki-67, OPN, OCN, DMP1, VDR, CYP24, CYP27B1, Endotelin1, VEGF165, and VEGF 189.

12. A method of enhancing osteogenic differentiation to treat bone defect with one-step surgical procedure, comprising the steps of:
isolating adipose tissue from a subject by liposuction;
separating adipose-derived stem cells from the adipose tissue;
pre-treating the separated adipose-derived stem cells for a predefined time 20 to 40 minutes with vitamin D3;
seeding the pre-treated stem cells onto one or more scaffolds;
washing of unattached stem cells from the scaffolds after a predefined time of 20 to 30 minutes;
culturing the stem cell seeded scaffold by utilizing a modified perfusion bioreactor to form a tissue-engineered construct, and
implanting the tissue-engineered construct into the subject without a need to obtain an autologous bone graft.

13. The method of claim 12, wherein the adipose tissue is a human adipose tissue.

14. The method of claim 12, wherein the vitamin D3 is calcitriol.

15. The method of claim 12, wherein the bioreactor comprises:
a chamber including an inlet channel coupled to a peristaltic pump;
a flow distributor disposed within the chamber comprises at least five distributor channels, the flow distributor is in fluid communication with the inlet channel;
a suction tube, and
a syringe filter,
wherein the flow distributor is configured to after flow-induced shear stress on a culture medium passed through the at least five distributor channels.

16. The method of claim 12, wherein the step of culturing, comprises: applying flow-induced shear stress on the stem cells seeded scaffolds.

17. The method of claim 12, wherein the plurality of human adipose stem cells is pre-treated with calcitriol of 10 nM for 30 mins.

18. The method of claim 12, wherein a combined effect of vitamin D3 and flow-induced shear stress is configured to synergistically enhance the osteogenic and angiogenic differentiation of the adipose-derived stem cells.

19. The method of claim 12, wherein the step of pre-treating, comprises: treating the calcitriol treated cells with one or more markers selected from the group consisting of RUNX2, ALP, SPARC, ki-67, OPN, OCN, DMP1, VDR, CYP24, CYP27B1, Endotelin1, VEGF165, and VEGF 189.
